(19) United States
(12) Patent Application Publication
(10) Pub. No.: US 2007/0265447 A1
(43) Pub. Date: Nov. 15, 2007

(54) Process for the Preparation of Ziprasidone (5-[2-[4-(1,2-Benziosothiazol-3-Y1)-1-Piperazinyl]Ethyl]-6-Chloro-1,3-Dihydro-2H-Indol-2- One

(76) Inventors: Carlos Zetina-Rocha, Burnaby (CA); Allan W. Rey, Brantford (CA); Stephen E. Horne, Burlington (CA); Matthew A. Buck, Mississauga (CA)

Correspondence Address: IVOR M. HUGHES, BARRISTER & SOLICITOR,;PATENT & TRADEMARK AGENTS, 175 COMMERCE VALLEY DRIVE WEST, SUITE 200, THORNHILL, ON, L3T 7P6, CA

(21) Appl. No.: 11/667,039

(22) PCT Filed: Nov. 4, 2005
(86) PCT No.: PCT/CA05/01721

§ 371 Date: May 4, 2007

(30) Foreign Application Priority Data
Nov. 5, 2004 (CA) 2487003

(51) Int. Cl. C07D 401/00

(52) U.S. Cl. 544/368

(57) ABSTRACT

The present invention provides a new and useful process for preparing 5-[2-[4-(1,2-benzisothiazol-3-yl)-1-piperazinyl]ethyl]-6-chloro-1,3dihydro-2H-indol-2-one (ziprasidone) and methods for its purification. The process comprises the steps of: (i) mixing 6-chloro-5-(2-chloroethyl)-1,3-dihydro-2H-indol-2-one with either a free base or salt form of 3-(1-piperazinyl)-1,2-benzoisothiazole, in the presence of an alkaline compound and a high-boiling polar organic solvent or mixture of high boiling polar organic solvents, (ii) heating the mixture and stirring for a sufficient amount of time to obtain ziprasidone formation, (iii) cooling the mixture, adding it to water and filtering off the product, (iv) adding water to the product and stirring the suspension, and (v) isolating crude ziprasidone.

### BACKGROUND OF THE INVENTION

This invention relates to a novel process for the preparation of ziprasidone (5-[2-[4-(1,2-benzisothiazol-3-yl)-1-piperazinyl]ethyl]-6-chloro-1,3-dihydro-2H-indol-2-one) of formula I. Some salts of ziprasidone, and in particular, its hydrochloride salt is a potent commercial antipsychotic agent useful in the treatment of various disorders, including schizophrenia and anxiety diseases. Ziprasidone hydrochloride is currently marketed under the proprietary name of Geodon. Other salts of ziprasidone are also reported to be effective for the treatment of the same type of diseases, for instance see Canadian patent 2,252,898 which describes a maleate salt.

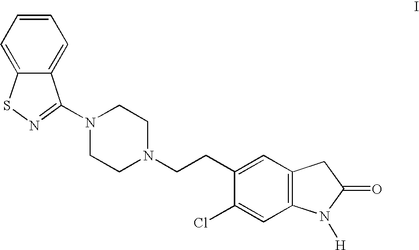

Thus, ziprasidone is a valuable precursor for the preparation of various salts with important pharmacological properties and commercial importance. Examples of current methods for the preparation of ziprasidone are described in U.S. Pat. Nos. 5,338,846, 5,312,925, 4,831,031; Canadian Patent No. 2,166,203; and PCT Application No. WO 2004/050655 and references cited therein.

Some of the processes described in the aforementioned patents necessitate the use of ion-exchange catalyst (i.e. sodium iodide) and/or phase transfer catalysts (for example tetra butyl ammonium bromide or tetra butyl phosphonium bromide) in order for the coupling reaction producing ziprasidone to take place. For example, U.S. Pat. No. 4,831,031 indicates that arylpiperazinyl-ethyl (or butyl)-heterocyclic compounds may be prepared by reacting piperazines of the formula II with compounds of the formula III as follows:

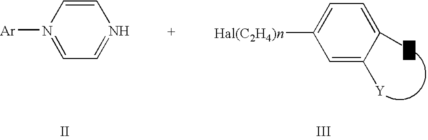

Wherein Hal is fluoro, chloro, bromo or iodo; and Ar, n, X and Y are as defined therein with reference to formula I. According to the '031 patent the coupling reaction is generally conducted in a polar solvent, such as a lower alcohol, dimethylformamide or methylisobutylketone, and in the presence of a weak base and that, preferably, the reaction is in the further presence of a catalytic amount of sodium iodide, and a neutralizing agent for hydrochloride such as sodium carbonate.

In some instances, the ziprasidone obtained in those manners was purified by column chromatography, thus making the process impractical for large-scale preparations. Another process uses potentially explosive gases such as hydrogen in the presence of catalysts, for example zinc, palladium on carbon, followed by acid treatment to carry out a reduction and cyclization of an intermediate, in order to obtain ziprasidone. Other processes utilize very large volumes of solvents such as tetrahydrofuran to accomplish the clarification and purification of crude ziprasidone (nearly 40 times the amount of crude ziprasidone, i.e. 40 volumes), thus severely limiting the utility of the process for large-scale manufacturing purposes.

The present invention provides a process for the preparation of ziprasidone in high yields and purity, suitable for large-scale manufacturing, which helps to overcome some of the deficiencies of the prior art.

### SUMMARY OF THE INVENTION

This invention relates to a process for the preparation of 5-[2-[4-(1,2-benzisothiazol-3-yl)-1-piperazinyl]ethyl]-6-chloro-1,3-dihydro-2H-indol-2-one, also known as ziprasidone, of formula I. The present invention further relates to a processes for the purification of crude ziprasidone.

Surprisingly, we have found that by using specific solvents, typically high-boiling polar organic solvents, we were able to produce pharmaceutical grade ziprasidone in an efficient, high-yielding manner.

Thus, in accordance with an aspect of the present invention there is provided a novel process for preparing ziprasidone comprising the steps of:
(i) mixing from about 1 to about 1.2 moles of 6-chloro-5-(2-chloroethyl)-1,3-dihydro-2H-indol-2-one and about 1 to about 1.2 moles of 3-(1-piperazinyl)-1,2-benzoisothiazole (in either its free-base form or a salt form, such as its hydrochloride salt) with about 2 to about 4 moles of an alkaline compound in about 3 to about 20 volumes of organic solvent or mixture of solvents,
(ii) heating the mixture at a temperature of from about 80 to about 140° C., until the reaction is complete,
(iii) cooling the mixture and adding it to water,
(iv) filtering, adding water to the solid and stirring,
(v) filtering and washing the solid,

The crude ziprasidone obtained by the above process, can then be dried or optionally, purified by:
(vi) stirring the solid with a suitable organic solvent or mixture of solvents at a temperature of between about 20 and 120° C.,
(vii) filtering and washing the solid,
(viii) drying the solid.

In another embodiment of this invention, a process is provided for the purification of ziprasidone by utilizing low volumes of solvent and comprising of the following steps:
(ix) dissolving the solid in 2 to 10 volumes of acetic acid or a polar organic solvent at a temperature of about 20 to about 80° C.,
(x) optionally filtering the solution,
(xi) precipitating the product by adding a suitable organic solvent or mixture of solvents at a temperature of about 20 to about 60° C.,
(xii) filtering, adding water or a suitable organic solvent to the solid and stirring at a temperature between about 40 to about 120° C.,
(xiii) filtering and washing the solid,
(xiv) drying the solid, or
(xv) repeating the above steps if desired.

Examples of organic solvents which are useful in the reaction of the present invention include, poly(ethylene glycol), poly(ethylene glycol) methyl ether, cyclic or acyclic amides such as 1-methyl-2-pyrrolidinone (NMP); dialkyl sulfones such as tetramethylene sulfone (sulfolane), and their mixtures thereof. The most preferred solvent is poly(ethylene glycol) methyl ether. Noteworthy is that the use of catalysts such as sodium iodide, when using those selected solvents, is not required.

The alkaline compound includes, but it is not limited to, sodium carbonate, potassium carbonate, lithium carbonate, sodium bicarbonate, potassium bicarbonate and the like.

Examples of suitable organic solvents, which are useful for the purification of crude ziprasidone in steps vi and vii, include but are not limited to, C1 to C6 alkanols such as methanol, ethanol, isopropanol and n-butanol.

Examples of suitable organic solvents, which are useful for the dissolution of ziprasidone in step ix, include but are not limited to 1-methyl-2-pyrrolidinone (NMP) and acetic acid. The most preferred solvent is acetic acid. The preferred volumes for the dissolution are about 2 to about 10 volumes of solvent.

Examples of suitable organic solvents, which are useful for the precipitation of ziprasidone in step xi, include but are not limited to C3 to C10 alkyl ethers such as methyl t-butyl ether, C5 to C10 alkanes such as hexanes, heptanes, cyclohexane, C1 to C6 alcohols such as ethanol and isopropanol; and their mixtures thereof.

Examples of suitable organic solvents, which are useful for the purification of ziprasidone in step xii and xiii, include but are not limited to C1 to C6 alcohols such as ethanol, isopropanol and n-butanol and their mixtures thereof.

In a preferred embodiment of the present invention about 1 to about 1.2 moles, more preferably about 1 to about 1.1 moles of 6-chloro-5-(2-chloroethyl)-1,3-dihydro-2H-indol-2-one and about 1 to about 1.2 moles, more preferably about 1 to about 1.1 moles of 3-(1-piperazinyl)-1,2-benzoisothiazole hydrochloride and about 2 to about 4 moles, more preferably about 2 to about 3 moles of sodium carbonate are used for the process of this invention.

The solvent volume required for step i is about 2 to 20 volumes, more preferably 3 to 8 volumes, most preferably about 4 to about 5 volumes. The most preferred solvent is poly(ethylene glycol) methyl ether or sulfolane, more preferably poly(ethylene glycol) methyl ether at a temperature of about 100 to 140° C.

In accordance with another aspect of this invention, the purification at step (vi) of ziprasidone involves stirring the product with 8 to 15 volumes, more preferably 10 to 12 volumes, of ethanol, isopropanol or n-butanol, more preferably ethanol at reflux temperature, then cooling and filtering the product.

In another prefer embodiment of the present invention ziprasidone is purified and clarified by dissolving the solid in 2 to 10 volumes, more preferably 2 to 6 volumes, of acetic acid at a temperature between about 20 and 80° C., more preferably between 40 and 60° C. and precipitating the product by adding an antisolvent or mixture of antisolvents. Examples of antisolvents include, but are not limited to C4 to C10 alkyl ethers such as diethyl ether or methyl t-butyl ether; or C5 to C10 alkanes such heptanes; or C1 to C6 alcohols such as ethanol; or their mixtures thereof. The most preferred antisolvents are methyl t-butyl ether, mixtures of ethanol-heptanes and mixtures of isopropanol-heptanes.

### DETAILED DESCRIPTION OF THE INVENTION

The following examples illustrate the preparation of ziprasidone and are not to be construed as limiting the scope of the invention in any manner.

### EXAMPLE 1

### Preparation of crude 5-[2-[4-(1,2-benzisothiazol-3-yl)-1-piperazinyl]ethyl]-6-chloro-1,3-dihydro-2H-indol-2-one

To a flask equipped with mechanical stirrer, thermometer, condenser and nitrogen inlet was added 6-chloro-5-(2-chloroethyl)-1,3-dihydro-2H-indol-2-one (21.6 g, 94 mmol), 3-(1-piperazinyl)-1,2-benzoisothiazole hydrochloride (24 g, 94 mmol), sodium carbonate (29.9 g, 282 mmol) and 1-methyl-2-pyrrolidinone (NMP) (96 mL) and the mixture was heated to 130-135° C. under nitrogen for about 24 hrs. The mixture was cooled to 40-45° C. and poured into water. The suspension was cooled and the product was collected by filtration on a Buchner funnel, the filter cake was rinsed with water at 20-25° C. and the damp product was transferred to a drying oven and dried in vacuo. This afforded 34.2 g (88.2% yield) of crude ziprasidone. The IR (KBr) and NMR spectra were consistent with those of reference ziprasidone.

### EXAMPLE 2

### Preparation of 5-[2-[4-(1,2-benzisothiazol-3-yl)-1-piperazinyl]ethyl]-6-chloro-1, 3-dihydro-2H-indol-2-one

To a flask equipped with mechanical stirrer, thermometer, condenser and nitrogen inlet was added 6-chloro-5-(2-chloroethyl)-1,3-dihydro-2H-indol-2-one (9.0 g, 39.1 mmol), 3-(1-piperazinyl)-1,2-benzoisothiazole hydrochloride (10.0 g, 39.1 mmol), sodium carbonate (9.96 g, 117.5 mmol) and poly(ethylene glycol) methyl ether (Mₙ=350, 40 mL) and the suspension was heated to 120-125° C. under nitrogen for about 48 hrs. The suspension was cooled and poured into water. The suspension was cooled to 20-25° C., the product was collected by filtration on a Buchner funnel and the filter cake was rinsed with water at 20-25° C. The damp product was transferred to a flask equipped with mechanical stirrer, 100 mL of water were added and the suspension stirred at ambient temperature for 1 h. The suspension was filtered, washed with water and transferred to a drying oven and dried in vacuo. This afforded 14.2 g (88% yield) of crude ziprasidone.

### EXAMPLE 3

### Purification of 5-[2-[4-(1,2-benzisothiazol-3-yl)-1-piperazinyl]ethyl]-6-chloro-1, 3-dihydro-2H-indol-2-one

To a flask equipped with mechanical stirrer, thermometer, condenser and nitrogen inlet was added crude ziprasidone (10.0 g, water damp, LOD=5.6%) and ethanol (120 mL) and the suspension was heated to reflux. The mixture was cooled and the product was collected by filtration in a Buchner funnel. The filter cake was rinsed with ethanol and transferred to a drying oven and dried to afford 7.8 g ziprasidone (83% recovery) having a 98.9% purity by HPLC.

### EXAMPLE 4

### Clarification-purification of 5-[2-[4-(1,2-benzisothiazol-3-yl)-1-piperazinyl]ethyl]-6-chloro-1,3-dihydro-2H-indol-2-one

To a flask equipped with mechanical stirrer, thermometer, condenser and nitrogen inlet was added ziprasidone produced as in the previous example (10.0 g) and acetic acid (20 mL) and the mixture was heated to dissolution. The hot solution was filtered through a Buchner funnel packed with a small amount of celite and then rinsed with 10 mL hot acetic acid. The filtrate was cooled and a mixture of heptanes and isopropanol 1:1 was added. The mixture was cooled to 20-25° C. and stirred and the product was collected by filtration in a Buchner funnel. The filter cake was rinsed with a mixture heptanes and isopropanol 1:2. The damp product was transferred to a flask equipped with mechanical stirrer and 100 mL of water were added and the suspension heated to 90-95° C. for 1 h. The suspension was cooled to 45-50° C., filtered, and washed with water. The damp product was transferred to a drying oven and dried in vacuo. This afforded 8.6 g (86% yield) of ziprasidone, 99.7% pure by HPLC.

While the foregoing provides a detailed description of the preferred embodiments of the invention, it is to be understood that the descriptions are illustrative only of the principles of the invention and not limiting. Furthermore, as many changes can be made to the invention without departing from the scope of the invention, it is intended that all material contained herein be interpreted as illustrative of the invention and not in a limiting sense.

1. A process for the preparation of ziprasidone comprising the steps of:
(i) mixing 6-chloro-5-(2-chloroethyl)-1,3-dihydro-2H-indol-2-one with either a free base or salt form of 3-(1-piperazinyl)-1,2-benzoisothiazole, in the presence of an alkaline compound and a high-boiling polar organic solvent or mixture of high-boiling polar organic solvents,
(ii) heating the mixture and stirring for a sufficient amount of time to obtain ziprasidone formation,
(iii) cooling the mixture, adding water to said mixture and filtering off the product,
(iv) adding water to the product and stirring the suspension, and
(v) isolating crude ziprasidone.

2. The process according to claim 1 further comprising the steps of:
(i) stirring said crude ziprasidone with a suitable organic solvent or mixture of solvents at a temperature of between about 20 to about 120° C.,
(ii) filtering and washing the resulting product, and
(iii) optionally drying the product.

3. A process for the purification of ziprasidone comprising the steps of:
(i) dissolving ziprasidone in acetic acid or a polar organic solvent at a temperature of about 20 to 80° C.,
(ii) optionally filtering the solution,
(iii) adding a suitable organic solvent or mixture of solvents at a temperature of about 20 to about 60° C. to precipitate the product,
(iv) filtering off the product, adding water or a suitable organic solvent to the product and stirring at a temperature between about 40 to about 120° C.,
(v) isolating the product, and
(vi) optionally drying the product.

4. The process of claim 1 or 2 wherein the high-boiling polar organic solvent is selected from poly(ethylene glycol), poly(ethylene glycol) methyl ether, 1-methyl-2-pyrrolidinone (NMP), and tetramethylene sulfone (sulfolane) and mixtures thereof.

5. The process of claim 1 or 2 wherein the alkaline compound is selected from sodium carbonate, potassium carbonate, lithium carbonate, sodium bicarbonate, potassium bicarbonate and the like.

6. The process of claim 1 or 2 wherein the 6-chloro-5-(2-chloroethyl)-1,3-dihydro-2H-indol-2-one is in a stoichiometric ratio of 0.8 to 1.2 with respect to 3-(1-piperazinyl)-1,2-benzoisothiazole hydrochloride.

7. The process of claim 1 or 2 wherein the alkaline compound is in a stoichiometric ratio of 2 to 4 with respect to 3-(1-piperazinyl)-1,2-benzoisothiazole hydrochloride.

8. The process of claim 5 wherein the alkaline compound is in a stoichiometric ratio of 2 to 4 with respect to 3-(1-piperazinyl)-1,2-benzoisothiazole hydrochloride.

9. The process of claim 2 or 4 wherein the organic solvent is selected from methanol, ethanol, isopropanol, n-butanol, and mixtures thereof, and said solvent containing water or water-free.

10. The process of claim 5 wherein the organic solvent is selected from methanol, ethanol, isopropanol, n-butanol, and mixtures thereof, and said solvent containing water or water-free.

11. The process of claim 6 wherein the organic solvent is selected from methanol, ethanol, isopropanol, n-butanol, and mixtures thereof, and said solvent containing water or water-free.

12. The process of claim 7 wherein the organic solvent is selected from methanol, ethanol, isopropanol, n-butanol, and mixtures thereof, and said solvent containing water or water-free.

13. The process of claim 8 wherein the organic solvent is selected from methanol, ethanol, isopropanol, n-butanol, and mixtures thereof, and said solvent containing water or water-free.

14. The process of claim 3 wherein the polar organic solvent is 1-methyl-2-pyrrolidinone (NMP).

15. The process of claim 3 or 14 wherein the organic solvent for step (iii) is selected from methyl t-butyl ether, hexanes, heptanes, cyclohexane, ethanol, isopropanol and mixtures thereof.

16. The process of claim 3 or 14 wherein the organic solvent for step (iv) is selected from ethanol, isopropanol, n-butanol and mixtures thereof.

17. The process of claim 15 wherein the organic solvent for step (iv) is selected from ethanol, isopropanol, n-butanol and mixtures thereof.
